(19) United States
(12) Patent Application Publication
(10) Pub. No.: US 2026/0283326 A1
(43) Pub. Date: Sep. 24, 2026

(54) MULTIFUNCTIONAL COMB AND METHOD OF TREATING SCALP USING THE SAME

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN); Mou Xiong, Shenzhen (CN); Tang Yuanzhou, Shenzhen (CN)

(73) Assignee: SHENZHEN NUON MEDICAL EQUIPMENT CO., LTD., Shenzhen (CN)

(21) Appl. No.: 19/277,480

(22) Filed: Jul. 23, 2025

(30) Foreign Application Priority Data
Mar. 21, 2025 (CN) 2025205120200

(51) Int. Cl. A45D 24/06; A45D 24/18; A45D 24/22; A61L 2/10; A61N 5/06; A61L 103/00

(52) U.S. Cl. CPC A45D 24/06; A45D 24/18; A45D 24/22; A61L 2/10; A61N 5/0617; A45D 2200/205; A61L 2103/00; A61L 2202/11; A61N 2005/0632; A61N 2005/0644; A61N 2005/0651

(57) ABSTRACT

A multifunctional comb is disclosed. The multifunctional comb comprises a first rotating arm that may include a plurality of first comb teeth and a first functional piece. A second rotating arm comprising a plurality of second comb teeth and a second functional piece. The second rotating arm is rotatably connected to the first rotating arm to form a folded state and an open state. In the folded state, the second rotating arm is folded relative to the first rotating arm, and in the open state, the second rotating arm is open relative to the first rotating arm. The first functional piece is configured to deliver a liquid to the scalp, and the second functional piece is configured to provide stimulation therapy to the scalp.

### TECHNICAL FIELD

The present invention relates generally to a personal care device and more particularly to a multifunctional comb and method of treating the scalp using the same.

### BACKGROUND

Combs are one of the most commonly used personal care tools for grooming hair. Combs are designed only for detangling or shaping hair strands and do not serve any therapeutic or skincare function. However, as people become more health-conscious and seek convenient ways to take care of their hair and scalp, the demand for multifunctional grooming devices has increased. In recent years, some devices have introduced features like scalp massage or vibration-based stimulation. However, these devices often come as separate devices, which require users to switch between tools or carry bulky equipment. Moreover, even when some features like liquid dispensing or light therapy are integrated into a single device, they tend to be non-portable, difficult to store, or not user-friendly. Their designs are often not foldable or compact, which makes them inconvenient for travel or daily use.

Additionally, most existing devices do not allow simultaneous scalp care during the natural act of combing, nor do they offer user-controlled dispensing mechanisms or safe, directed light treatment integrated into a compact form. The lack of integration between grooming and therapeutic features results in lost time, reduced convenience, and sub-optimal care routines. Fruthermore, existing comb devices lacks feature of providing two therapy simultaneously.

Therefore, there is a need for a lightweight, portable, and user-friendly multifunctional comb that allows a user to comb their hair while also delivering scalp treatments, without the need for separate equipment or complex steps, and is able to provide multiple therapy simultaneously.

### OBJECTS OF THE INVENTION

An objective of the present invention is to provide a multifunctional comb that allows a user to simultaneously comb hair and treat the scalp, thereby improving convenience and efficiency in personal grooming and care routines.

Another objective of the present invention is to provide a multifunctional comb that integrates a first functional piece configured to dispense liquid, such as hair care serum or tonic, directly onto the scalp via liquid guide columns, enabling targeted and controlled application of treatment solution during combing.

Another objective of the present invention is to provide a multifunctional comb having a second functional piece that includes a lamp board and light source capable of delivering stimulation therapy, such as phototherapy, microcurrent, iontophoresis, massage, hot or cold sensation through components like a lamp board and light-emitting diodes (LEDs), heating element, cooling element, , microcurrent module etc.

A further objective of the present invention is to improve the hygiene of the comb during storage by integrating a UV disinfection module that is automatically activated when the comb is folded, effectively sanitizing liquid-contact components and comb teeth.

An additional objective of the present invention is to utilize infrared, red, blue, or ultraviolet LEDs selected for therapeutic and antimicrobial properties, thereby offering tailored scalp treatments and enhancing device hygiene through scientifically proven light frequencies.

Yet another objective of the present invention is to provide a compact and portable comb design, wherein a first rotating arm and a second rotating arm are rotatably connected, allowing the comb to be folded for storage or travel and unfolded for use, thereby reducing space requirements and enhancing portability.

Another objective of the present invention is to provide a structural arrangement in which critical components such as the battery, lamp board, and light source are stacked and supported efficiently within the second arm, maintaining a slim form factor while ensuring functional reliability.

Another objective of the present invention is to enable intuitive operation of the stimulation therapy function through a pressing switch and charging columns, providing seamless user control of light activation and safe battery charging with minimal surface intrusion.

### SUMMARY OF THE INVENTION

In an embodiment, a multifunctional comb is disclosed. The multifunctional comb may include a first rotating arm that may include a plurality of comb teeth and a first functional piece. The multifunctional comb may further include a second rotating arm that may include a plurality of second comb teeth and a second functional piece. In an embodiment, the second rotating arm is rotatably connected to the first rotating arm to form a folded state and an open state. In an embodiment, in the folded state, the second rotating arm may be folded relative to the first rotating arm, and in the open state, the second rotating arm may be open relative to the first rotating arm. In an embodiment, the first functional piece may be configured to deliver a liquid to a scalp, and the second functional piece may be configured to provide stimulation therapy to the scalp.

In accordance with the embodiment, the second functional piece is selected from a group consisting of, but not limited to a microcurrent module, a steamer module, a phototherapy module, an iontophoresis module, and a massage module. In an embodiment, the first rotating arm may include a reservoir chamber, and the first functional piece may include a plurality of liquid guide columns and an occlusion piece. In an embodiment, one end of the plurality of liquid guide columns may be fixed to the first rotating arm, and the other end protrudes outside the first rotating arm. In an embodiment, each of the plurality of liquid guide columns has a liquid conduction channel in fluid communication with the reservoir chamber, and the occlusion piece is configured to selectively block and open the liquid conduction channel.

In accordance with the embodiment, the first rotating arm may include a first shell, a second shell, and a comb tooth piece. In an embodiment, the first shell and the second shell may define a first storage cavity, and the second shell may include a plurality of first relinquishing holes, and a plurality of second relinquishing holes. In an embodiment, the comb tooth piece may include an integrally formed connecting portion and the plurality of first comb teeth, the connecting portion being disposed within the first storage cavity, and the plurality of first comb teeth extending through the plurality of first relinquishing holes. In an embodiment, the plurality of liquid guide columns may extend through the plurality of second relinquishing holes and may be provided with limiting flanges that may be clamped between the second shell and the connecting portion.

In accordance with the embodiment, one side of each of the plurality of second relinquishing holes opposite the connecting portion may be provided with a ring of retaining walls, an end of the ring of retaining walls may be sealed against an inner wall of the first shell to form the reservoir chamber. In an embodiment, the first shell may further include a liquid injection port in communication with the reservoir chamber, the liquid injection port being covered by a detachable cover plate.

In accordance with the embodiment, the second functional piece may include a lamp board and at least one light source disposed on the lamp board.

In accordance with the embodiment, the second rotating arm may include a third shell and a fourth shell forming a second storage cavity that may house a battery and the second functional piece. In an embodiment, the fourth shell may include a light transmission hole corresponding to the at least one light source, and a light transmission plate may be disposed over the light transmission hole on exterior side of the fourth shell.

In accordance with the embodiment, the battery and the lamp board may be stacked along the thickness direction of the second rotating arm.

In accordance with the embodiment, the inner wall of the second rotating arm may be provided with a plurality of support columns. In an embodiment, the plurality of support columns may surround the light transmission hole and the lamp board and may be configured to support the battery.

In accordance with the embodiment, the outer surface of the second rotating arm may include a groove corresponding to each of the support columns, and an end of each second comb tooth may be inserted into the groove.

In accordance with the embodiment, the multifunctional comb may further include a pressing switch and two charging columns. In an embodiment, the pressing switch may include two perforations through which the two charging columns pass. In an embodiment, the pressing switch is configured to trigger a switching device on the lamp board, and the charging columns are electrically connected to the lamp board.

In another embodiment, the multifunctional comb is disclosed. The multifunctional comb may include a first rotating arm that may include a plurality of comb teeth. The multifunctional comb may further include a second rotating arm that may include a plurality of second comb teeth. The multifunctional comb may further include at least one functional piece in the first rotating arm or the second rotating arm. In an embodiment, the second rotating arm is rotatably connected to the first rotating arm to form a folded state and an open state. In an embodiment, in the folded state, the second rotating arm may be folded relative to the first rotating arm, and in the open state, the second rotating arm may be open relative to the first rotating arm. The at least one functional piece is configured to provide stimulation therapy to the scalp and is activated upon detecting that the multifunctional comb is in the open state.

In certain embodiments, the second functional piece may further include a UV light source configured to be automatically activated in the folded state for disinfection purposes.

In some embodiments, the method may further include transitioning the multifunctional comb into a folded state to automatically activate the UV light source for a predefined duration. The UV light source may disinfect the comb teeth and liquid-contact areas during storage, thereby reducing microbial contamination and enhancing hygiene.

In another embodiment, a method of treating a scalp using a multifunctional comb is disclosed. The method may include unfolding the multifunctional comb from a folded state to an open state, wherein the multifunctional comb comprises a first rotating arm and a second rotating arm, wherein the first rotating arm is rotatably connected to the second rotating arm. The method may further include applying a plurality of first comb teeth of the first rotating arm and a plurality of second comb teeth of the second rotating arm to the scalp. The method may further include dispensing a liquid from a reservoir chamber in the first rotating arm through a plurality of liquid guide columns toward the scalp. The method may further include activating a light source disposed on a lamp board within the second rotating arm to emit phototherapy light toward the scalp.

In accordance with the embodiment, the dispensing of the liquid may include contacting an occlusion piece disposed at end of each of a plurality of liquid guide columns to selectively open a liquid conduction channel in fluid communication with the reservoir chamber.

In accordance with the embodiment, each liquid guide column from the plurality of liquid guide columns may be supported by a second shell of the first rotating arm and may extend through a relinquishing hole and may further include a limiting flange clamped between the second shell and a connecting portion of a comb tooth piece.

In accordance with the embodiment, the reservoir chamber may be formed between a ring of retaining walls and an inner wall of a first shell of the first rotating arm, and the reservoir chamber may be refillable via a liquid injection port sealed by a detachable cover plate.

In accordance with the embodiment, the light source is disposed on the lamp board and may emit through a light transmission hole in a fourth shell of the second rotating arm.

In accordance with the embodiment, the light transmission hole may be covered by a light transmission plate disposed on exterior surface of the second rotating arm.

In accordance with the embodiment, the lamp board and a battery supplying power to the light source may be stacked along the thickness direction of the second rotating arm.

In accordance with the embodiment, the battery may be supported by a plurality of support columns may surround the light transmission hole and may be disposed within a second storage cavity of the second rotating arm.

In accordance with the embodiment, end of the plurality of second comb teeth may be inserted into grooves in the outer surface of the second rotating arm, each groove corresponding to one of the support columns.

In accordance with the embodiment, the method may further include pressing a pressing switch to trigger a switching device on the lamp board and charging the battery via two charging columns aligned with perforations in the pressing switch.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first’, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term "several" means more than one, unless otherwise specified.

In the context of the specification, the term "LED module" refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of this specification, terms like “light,” “radiation,” “irradiation,” “emission” and “illumination,” etc. refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorised into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for the treatment of conditions that occur at the skin surface, such as psoriasis or infection.

In the context of the specification, the term “light source” or “phototherapy source” etc. refers to a source emitting coherent laser light, or light-emitting diodes (“LEDs”). The term “light therapy” refers to light generated from any of the source, such as lasers, LED sources or Super luminous diodes (“SLD”). In certain embodiments, the light source may include UV LEDs for non-therapeutic disinfection purposes when the comb is in a folded or storage state.

In the context of the specification, the term "occlusion piece" refers to a movable component, such as a valve or spring-loaded element, disposed at the outlet of the liquid guide column and configured to block or permit fluid flow based on contact with the scalp or user-applied pressure.

In the context of the specification, the term "lamp board" refers to a printed circuit board or similar substrate on which one or more light sources are mounted and electrically connected to a power source, such as a rechargeable battery.

In the context of the specification, the term "light source" refers to any active component, such as a light-emitting diode (LED), capable of emitting phototherapy light (e.g., red, infrared, or blue) toward the scalp.

In the context of the specification, the term "light transmission hole" refers to an aperture formed in the housing of the second rotating arm, aligned with the light source to allow therapeutic light to exit the comb and reach the scalp.

In the context of the specification, the term "light transmission plate" refers to a transparent or translucent cover positioned over the light transmission hole to protect internal components while allowing light to pass through.

In the context of the specification, the term "pressing switch" refers to a mechanical actuator disposed on the surface of the comb and configured to trigger an internal switching device that activates the lamp board or associated electronics.

In the context of the specification, the term “plurality” means two or more than two, unless otherwise indicated.

In the context of the specification, the term "several" means more than one, unless otherwise specified.

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colours), Surface Mount Technology (SMT) LEDs, Bi-colour LEDs, Pulse Width Modulated RGB (Red-Green-Blue) LEDs, and high-power LEDs, among others. The LEDs may also include UV-emitting diodes specifically selected for antimicrobial action in disinfection modules integrated into the multifunctional comb.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminium, Boron, Zinc Selenide, etc. in pure form or doped with elements such as Aluminium and Indium etc. For example, red and amber colours are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider colour gamut. White and other colored lightings may also be produced using phosphor coatings such as Yttrium Aluminium Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

### BRIEF DESCRIPTION OF THE DRAWING

The accompanying drawings, which are incorporated in and constitute a part of this disclosure, illustrate exemplary embodiments and, together with the description, serve to explain the disclosed principles.

To more clearly illustrate the technical solutions described in the embodiments of the present invention, the accompanying drawings referenced below are provided as part of the detailed description. It should be understood that these drawings represent only certain exemplary embodiments of the invention, and that other configurations and variations may be derived from these illustrations by those skilled in the art without departing from the scope of the present disclosure.

FIG. 1 illustrates a perspective view of a multifunctional comb, in accordance with an embodiment of the present disclosure.

FIG. 2 illustrates a perspective view of the multifunctional comb of FIG. 1, in accordance with an embodiment of the present disclosure.

FIG. 3 illustrates an exploded view of the multifunctional comb of FIG. 1, in accordance with an embodiment of the present disclosure.

FIG. 4 illustrates a longitudinal sectional view of the multifunctional comb of FIG. 1, in accordance with an embodiment of the present disclosure.

FIG. 5 illustrates an exploded view of a second rotating arm showing the light source present in the multifunctional comb, in accordance with an embodiment of the present disclosure.

FIG. 6 illustrates another longitudinal sectional view of the multifunctional comb of FIG. 1, in accordance with an embodiment of the present disclosure.

FIG. 7 illustrates a traverse sectional view of the multifunctional comb of FIG. 1, in accordance with an embodiment of the present disclosure.

FIG. 8 illustrates a magnified view of section A in FIG. 6, in accordance with an embodiment of the present disclosure.

FIG. 9 illustrates a magnified view of section B in FIG. 6, in accordance with an embodiment of the present disclosure.

FIG. 10 is a flowchart of a methodology to treat the scalp using the multifunctional comb, in accordance with an embodiment of the present disclosure.

### DETAILED DESCRIPTION OF THE DRAWINGS

The foregoing description has broadly outlined the features and technical advantages of the present disclosure in order that the detailed description of the disclosure that follows may be better understood. Additional features and advantages of the disclosure will be described hereinafter which forms the subject of the claims of the disclosure. It should be appreciated by those skilled in the art that the conception and specific embodiments disclosed may be readily utilized as a basis for modifying other devices, systems, assemblies, and mechanisms for conducting the same purposes of the present disclosure. It should also be realized by those skilled in the art that such equivalent constructions do not depart from the scope of the disclosure as set forth in the appended claims. The novel features, which are believed to be characteristics of the disclosure, to its device or system, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

The technical solutions described in the embodiments of the present disclosure will be clearly and fully explained below with reference to the accompanying drawings. It should be understood that the embodiments provided herein are merely illustrative and do not represent the entirety of possible implementations. Variations and modifications to these embodiments that may be conceived by those skilled in the art, without departing from the spirit and scope of the invention and without the exercise of inventive faculty, are intended to fall within the scope of protection afforded by the present disclosure.

It should be noted that, in the description of the present invention, the terms "first," "second," and the like are used solely for the purpose of distinguishing between different elements and should not be construed as indicating any order, priority, or relative importance. Such terms are not intended to imply a specific sequence or quantity unless explicitly stated. Accordingly, references to “first” and “second” components may each refer to one or more of such components. Furthermore, the term "and/or" as used herein is intended to encompass any and all combinations of one or more of the associated listed items. For example, the expression “A and/or B” should be interpreted to include: A alone, B alone, or both A and B together.

The terms “including”, “comprises”, “comprising”, “comprising of” or any other variations thereof, are intended to cover a non-exclusive inclusion, such that a system or a device that comprises a list of components or steps does not include only those components or steps but may include other components or steps not expressly listed or inherent to such setup or device. In other words, one or more elements in a system or apparatus proceeded by “comprises… a” does not, without more constraints, preclude the existence of other elements or additional elements in the system or apparatus.

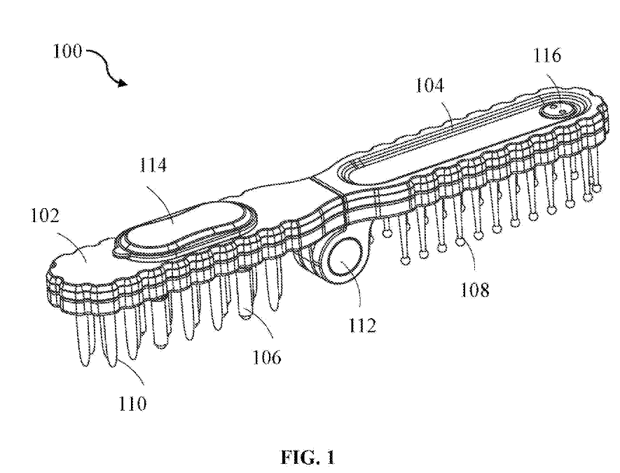

Reference will now be made to the exemplary embodiments of the disclosure, as illustrated in the accompanying drawings. Wherever possible, same numerals have been used to refer to the same or like parts. The following paragraphs describe the present disclosure with reference to FIGS. 1-10. As summarized above, in one broad aspect, the present invention provides a multifunctional comb and a method of treating the scalp using the same.

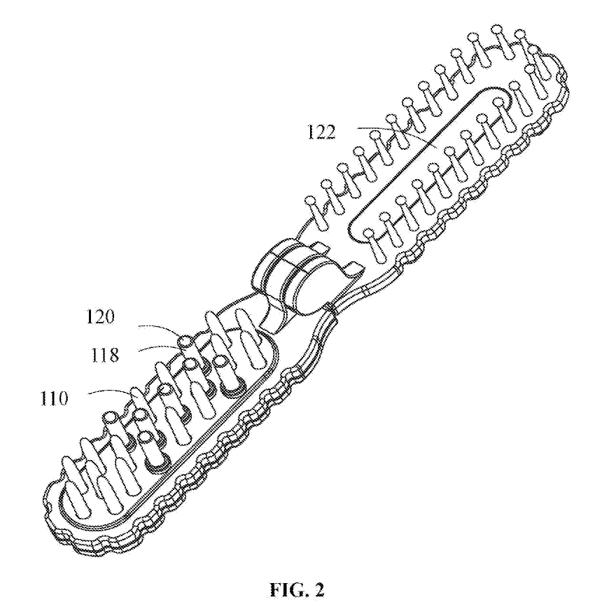

Referring to FIG. 1 and FIG. 2, a multifunctional comb 100 that may include a first rotating arm 102 and a second rotating arm 104 is illustrated. The second rotating arm 104 may be rotatably connected to the first rotating arm 102, such that the multifunctional comb 100 may transition between a folded state and an open state.

In the folded state, the second rotating arm 104 may be folded relative to the first rotating arm 102, thereby allowing the first rotating arm 102 and the second rotating arm 104 to be compactly arranged together. In the open state, the second rotating arm 104 may be unfolded outward relative to the first rotating arm 102, thereby enabling the multifunctional comb 100 to be used.

Specifically, a rotating shaft 112 may be disposed between the first rotating arm 102 and the second rotating arm 104 to facilitate the rotational connection and allow for folding and unfolding of the first rotating arm 102 and the second rotating arm 104.

The first rotating arm 102 may include a plurality of first comb teeth 110, and the second rotating arm 104 may include a plurality of second comb teeth 108. The first comb teeth 110 and the second comb teeth 108 are used for combing hair with therapeutic benefits. The shape and arrangement density of the plurality of first comb teeth 110 and the plurality of second comb teeth 108 may be configured differently in order to satisfy various usage requirements for different hair types or grooming purposes.

In the folded state, the plurality of first comb teeth 110 may be folded in the direction of the plurality of second comb teeth 108. The plurality of first comb teeth 110 and the plurality of second comb teeth 108 may be interleaved, wherein the plurality of first comb teeth 110 may be inserted into the gaps between the plurality of second comb teeth 108, thereby reducing the overall volume of the multifunctional comb for compact storage and portability. Alternatively, the plurality of first comb teeth 110 and the plurality of second comb teeth 108 may be folded in non-overlapping directions to suit particular folding configurations.

Moreover, the multifunctional comb 100 may be configured such that opening the multifunctional comb 100 from the folded state to the open state automatically enables activation of the first functional piece 106 and/or the second functional piece 157. The first rotating arm 102 and the second rotating arm 104 may be rotatably connected to a pivot axis defined by the rotating shaft 112. When the multifunctional comb 100 transitions into the open state, mechanical, magnetic, or optical detection elements positioned proximate to the rotating shaft may detect the relative positioning of the first rotating arm 102 and the second rotating arm 104.

Furthermore, an infrared (IR) sensor may be positioned within the housing of the first rotating arm 102 or the second rotating arm 104 near the rotating shaft 112. When the multifunctional comb 100 transitions from the folded state to the open state, the IR sensor detects the change in position or distance between the first rotating arm 102 and the second rotating arm 104, and generates a signal to activate the light source 159 on the lamp board 158.

Additionally, a pressure sensor may be arranged within the multifunctional comb 100, such that when the folding force is released and the comb is unfolded, the pressure sensor detects the reduction in compression and triggers activation of the phototherapy function or other operational modules.

In another embodiment, a mechanical switch or limit switch may be integrated into the hinge portion adjacent to the rotating shaft 112. The mechanical switch may be configured such that when the second rotating arm 104 is rotated outward beyond a predefined threshold angle relative to the first rotating arm 102, the mechanical switch closes an electrical circuit and automatically powers on the second functional piece 157.

Upon transitioning the multifunctional comb 100 back into the folded state, the IR sensor, the pressure sensor, or the hinge-based mechanical switch may detect the folded configuration and correspondingly deactivate the active modules, such as turning off the light source 159 or disabling liquid dispensing functionalities as applicable.

In some embodiments, the multifunctional comb 100 further includes an ultraviolet (UV) light source configured to perform disinfection of the multifunctional comb 100 surfaces, including areas that may be exposed to residual treatment liquid. The UV light source may be disposed within the first rotating arm 102, or the second rotating arm 104, or positioned adjacent to the rotating shaft 112. In particular, the UV light source may be positioned to irradiate the plurality of first comb teeth 110, the plurality of second comb teeth 108, and the surrounding surfaces of the first rotating arm 102 and the second rotating arm 104, where treatment liquid dispensed through the liquid guide columns 118 may accumulate. This configuration may be advantageous because, unlike conventional combs with UV lights merely for superficial disinfection, the multifunctional comb 100 may structurally separate the liquid dispensing components (housed in the first rotating arm 102) and the light source (housed in the second rotating arm 104).

Specifically, when the comb is in the folded or closed position, the UV light source housed within the lamp board 158 of the second rotating arm 104 becomes active and emits light directly onto the dispensing components or the first comb teeth 110 located in the first rotating arm 102. This setup improves the overall disinfection efficiency while also safeguarding the user's eyes by keeping the UV light contained within the device during operation. As a result, it effectively mitigates contamination risks arising from regular use.

Furthermore, the UV light may be activated only when the multifunctional comb 100 transitions into the folded state. A sensor or switch, such as an IR sensor, pressure sensor, or mechanical limit switch, may detect the closed configuration of the multifunctional comb 100 and trigger activation of the UV light source. Upon activation, the UV light source emits UV radiation for a predetermined time interval sufficient to disinfect the comb teeth and liquid-contacted surfaces without requiring user intervention. After the expiration of the predefined time, the UV light source may be automatically deactivated to prevent excessive UV exposure and to conserve power.

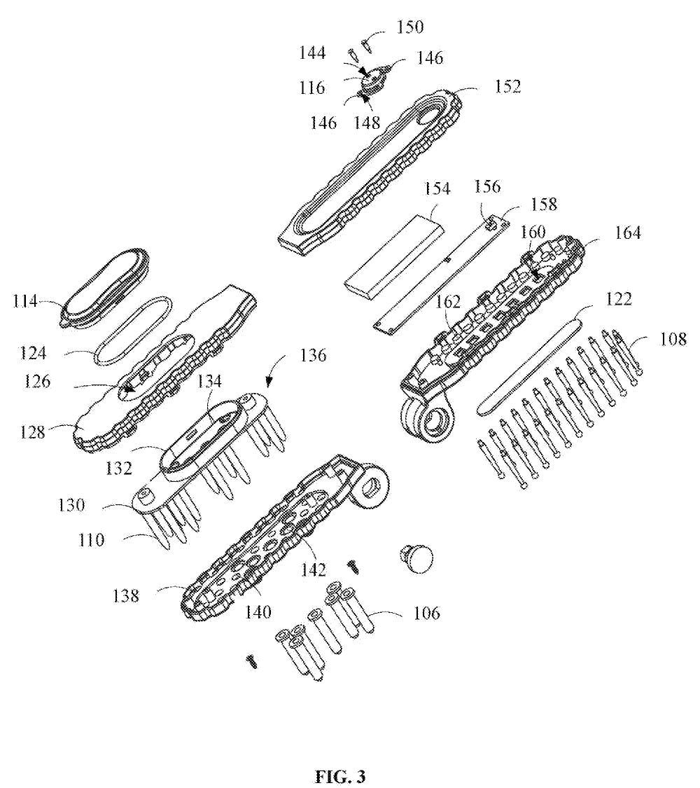

Referring to FIG. 3 to FIG. 6, the first rotating arm 102 may be provided with a first functional piece 106, and the second rotating arm 104 may be provided with a second functional piece 157. The first functional piece 106 and the second functional piece 157 may each correspond to one or more type of functional module, such as, but not limited to, a phototherapy module, a microcurrent module, an iontophoresis module, a fluid conduction module, or a massage module, a heating element, a cooling element, a steamer module, depending on the application requirements.

The phototherapy module, when implemented as the second functional piece 157, may include at least one light source 159 configured to emit therapeutic light, in visible or non-visible ranges, including red, blue, or infrared light, for performing phototherapy on the scalp or skin. The phototherapy may assist in effects such as anti-ageing, acne reduction, or anti-inflammatory treatment. Additionally, the phototherapy module incorporates a UV light source, specifically designed for disinfection or sterilization purposes.

In some embodiments, the second functional piece 157 may alternatively be implemented as a microcurrent module or an iontophoresis module, each of which includes two electrodes. When the electrodes come into contact with the skin, they form an electrical circuit with the scalp and the internal circuit board. In the case of a microcurrent module, the multifunctional comb 100 delivers pulsed stimulation to the scalp. In the case of an iontophoresis module, stimulation may be applied without pulses to promote transdermal delivery of active agents through the scalp.

In an embodiment, where the first functional piece 106 may be implemented as a fluid conduction module, the first functional piece 106 may include a reservoir chamber 134, configured to store a treatment liquid, such as hair care serums, medicaments, or a hair growth solution, etc. The stored liquid may be dispensed toward the scalp via a liquid conduction channel 170 formed within each liquid guide column 118, thereby enabling targeted and continuous delivery of the care solution during use.

In certain embodiments, the first functional piece 106 may further be implemented as a massage module. The massage module may include either a rotatable ball or a fixed bump. The rotatable ball may be configured to rotate relative to the first rotating arm 102, thereby enabling a rolling massage effect across the scalp. Alternatively, the fixed bump remains stationary relative to the first rotating arm 102 and may provide a pressing or squeezing massage effect during combing.

In an embodiment of the present invention, the effect of first functional piece 106 can be used by the second function piece 157. For example, when the first functional piece 106 is implemented as a phototherapy module, the second functional piece 157 is able to use the heat generated by the LEDs to impart heating function.

The multifunctional comb 100, according to the present embodiment, not only performs the basic function of combing hair but also enables simultaneous scalp and hair care through the use of the first functional piece 106 and the second functional piece 157. This configuration allows the user to receive scalp maintenance such as liquid application, phototherapy, or massage while combing, thereby enhancing the user experience and optimizing the use of time during grooming.

Further, because the first functional piece 106 and the second functional piece 157 may be mounted on separate arms, such as the first rotating arm 102 and the second rotating arm 104, they do not interfere with one another during operation. This independent arrangement provides the user with the flexibility to use either of the first functional piece 106 and the second functional piece 157, depending on the desired application, thus improving operational convenience.

However, integrating both the first functional piece 106 and the second functional piece 157 into a single device, such as the multifunctional comb 100, requires additional internal space, thereby increasing the overall size of the comb. This presents a challenge in terms of storage and portability. To address this issue, the multifunctional comb 100 may be designed such that the first rotating arm 102 and the second rotating arm 104 may be rotatably connected. As described in previous sections, the multifunctional comb 100 may be folded into a compact form for storage and unfolded during use, effectively overcoming the limitations of increased device size while maintaining full functionality.

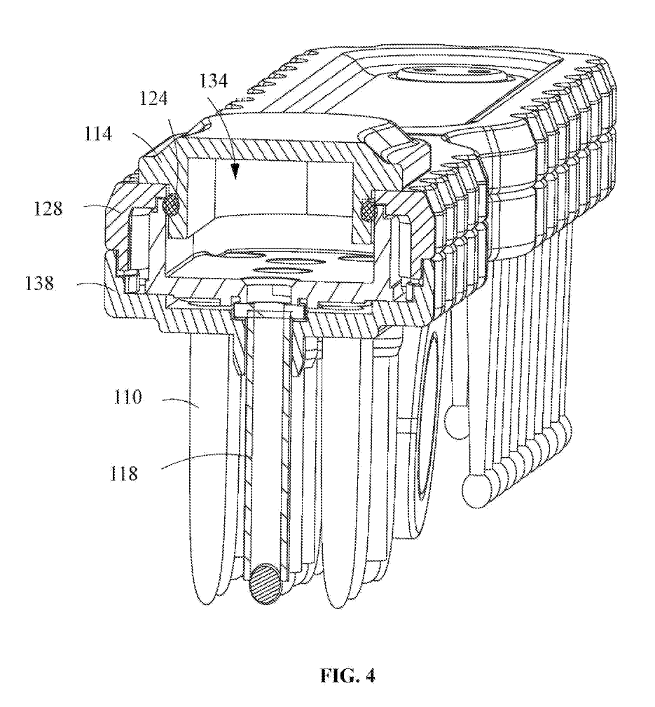

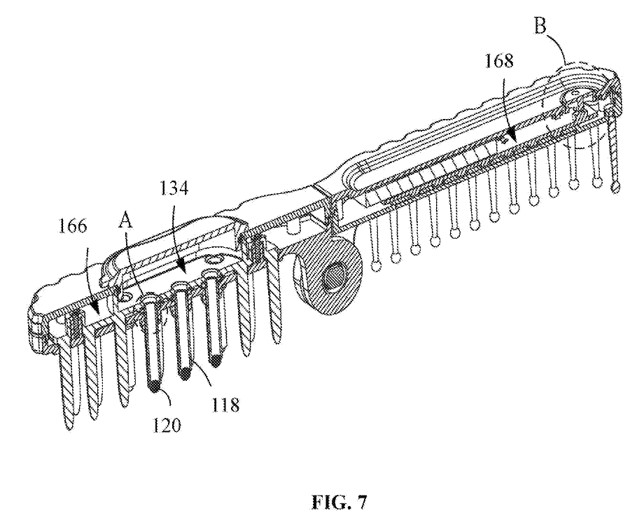

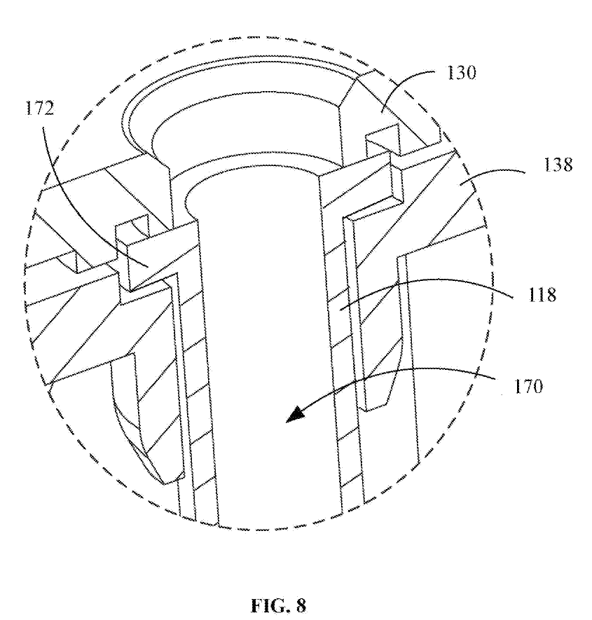

Referring to FIG. 4, FIG. 7, and FIG. 8, in some embodiments, the first functional piece 106 may be implemented as a fluid conduction module. Specifically, the first rotating arm 102 may include the reservoir chamber 134 configured to hold a liquid treatment solution, such as a scalp care formulation or hair growth serum. The first functional piece 106 may include a plurality of liquid guide columns 118 and an occlusion piece 120. One end of each of the plurality of liquid guide columns 118 may be fixed to the first rotating arm 102, while the other end protrudes externally beyond the surface of the first rotating arm 102 to enable direct contact with the scalp. This configuration ensures that the plurality of liquid guide columns 118 may not be obstructed by the surroundings during use, thereby allowing efficient liquid delivery.

The plurality of liquid guide columns 118 may define the liquid conduction channel 170 that may be in fluid communication with the reservoir chamber 134. The occlusion piece 120 may be configured to selectively open or block the liquid conduction channel 170. When the occlusion piece 120 may be in the open position, the care liquid stored within the reservoir chamber 134 may flow through the liquid conduction channel 170 and may be dispensed onto the scalp. When the occlusion piece 120 is in the closed position, the occlusion piece 120 may block the liquid conduction channel 170 to prevent unintended leakage of the liquid.

In an embodiment, the occlusion piece 120 may be implemented as a solenoid valve or a manual mechanical valve. Alternatively, the occlusion piece 120 may be configured as a spherical element positioned at one end of the liquid conduction channel 170, opposite the reservoir chamber 134. The liquid conduction channel 170 may be formed with a conical shape, where the larger end may be connected to the reservoir chamber 134, and the smaller end accommodates the occlusion piece 120. A portion of the occlusion piece 120 may protrude outward from the plurality of liquid guide columns 118 to enable physical engagement with the scalp.

A spring element may be positioned within the plurality of liquid guide columns 118, arranged to bias the occlusion piece 120 outward. When the occlusion piece 120 may be pressed inward, such as when the occlusion piece 120 may be in contact with the scalp, the spring element may be compressed, causing the liquid conduction channel 170 to open partially and allow the treatment liquid to flow from the reservoir chamber 134 toward the scalp. When external pressure may be removed (i.e., when the occlusion piece 120 may no longer be in contact with the scalp), the spring may automatically push the occlusion piece 120 back to its resting portion at the narrow end of the liquid conduction channel 170, thereby sealing the liquid conduction channel 170 and preventing unintended leakage of fluid.

In some embodiments, the plurality of liquid guide columns 118 and the plurality of first comb teeth 110 may be both disposed on the same side of the first rotating arm 102. This configuration may allow for simultaneous combing and scalp treatment, such as liquid dispensing, during normal grooming. Optionally, the plurality of liquid guide columns 118 may protrude further outward than the plurality of first comb teeth 110 to ensure that the occlusion piece 120 may make direct contact with the scalp without interference from the plurality of first comb teeth 110.

Referring to FIG. 3, FIG. 4, and FIG. 8, in some embodiments, the first rotating arm 102 may include a first shell 128, a second shell 138, and a comb tooth piece 136. The first shell 128 and the second shell 138 may cooperate to define a first storage cavity 166 that may house the internal components of the first rotating arm 102. The second shell 138 may be provided with a plurality of first relinquishing holes 140 and a plurality of second relinquishing holes 142. The comb tooth piece 136 may include an integrally formed connecting portion 130 and the plurality of first comb teeth 110. The connecting portion 130 may be positioned within the first storage cavity 166, and the plurality of first comb teeth 110 may extend through the corresponding first relinquishing hole 140 in the second shell 138 to protrude externally for combing.

The plurality of liquid guide columns 118 may also be arranged to extend through the corresponding second relinquishing hole 142 of the second shell 138. Each liquid guide column 118 may include a limiting flange 172 formed on its peripheral surface near the distal end. The limiting flange 172 may be jointly clamped between the second shell 138 and the connecting portion 130 of the comb tooth piece 136. This configuration mechanically secures the plurality of liquid guide columns 118 in place and restricts their longitudinal movement, thereby ensuring stable operation during scalp contact and liquid dispensing.

In this embodiment, the comb tooth piece 136 and the surrounding housing structure may include the first shell 128, and the second shell 138 may be configured to mechanically clamp the plurality of liquid guide columns 118. No additional fastening or support structure may be required to secure the plurality of liquid guide columns 118, thereby simplifying the internal construction and reducing manufacturing cost and complexity.

The connecting portion 130 of the comb tooth piece 136 may be disposed within the first storage cavity 166 and may be restrained against outward displacement by the second shell 138. The plurality of first comb teeth 110 may be integrally formed with the connecting portion 130 to constitute the comb tooth piece 136. This integrated design may facilitate one-step installation into the housing assembly and may eliminate the need for individual placement of separate comb teeth, thereby improving assembly efficiency.

In some embodiments, one side of the connecting portion 130 opposite to the plurality of second relinquishing holes 142 may be provided with a ring-shaped retaining wall 132. The end of the retaining wall 132 may be sealed against the inner surface of the first shell 128, thereby enclosing the reservoir chamber 134. The first shell 128 may further include a liquid injection port 126 in fluid communication with the reservoir chamber 134. The liquid injection port 126 may be covered by a detachable cover plate 114, which may allow the user to refill the reservoir chamber 134 with a liquid formulation, such as a scalp treatment or hair care solution.

The detachable cover plate 114 may be removable secured to the first shell 128 using various mechanisms, including buckle clamping, threaded screw engagement, or magnetic attachment, depending on the design requirements. A sealing ring 124 may be disposed between the detachable cover plate 114 and the first shell 128 to ensure a leak-proof seal and prevent fluid leakage during use or transport.

In some embodiments, the second functional piece 157 may be implemented as a phototherapy module. The second functional piece 157 may include a lamp board 158 and the at least one light source 159 disposed on the lamp board 158 and the at least one light source 159 disposed on the lamp board 158. The light source 159, may be configured to emit therapeutic light, such as red, infrared, or blue light, for application to the scalp during grooming.

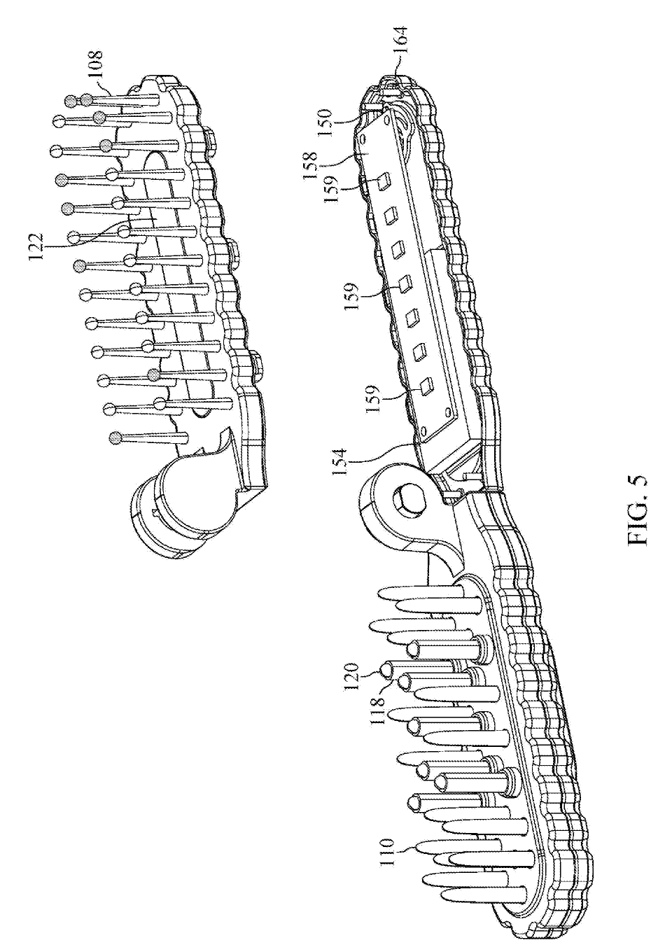

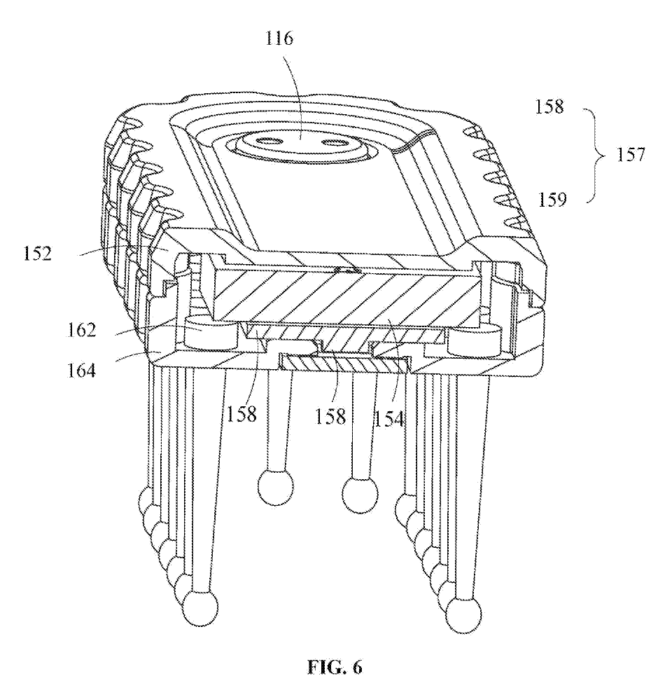

Referring to FIG. 3, FIG. 5, and FIG. 6, in some embodiments, the second functional piece 157 may be disposed within the second rotating arm 104. The second rotating arm 104 may include a third shell 152 and a fourth shell 164, which together enclose a second storage cavity 168. Within the second storage cavity 168, the second functional piece 157, implemented, for example, as a phototherapy module, may be arranged alongside a battery 154. The second functional piece 157 may include the lamp board 158 and the light source 159 mounted thereon. The battery 154 may be electrically connected to the lamp board 158 and may be configured to supply power to the light source 159 for operation.

The fourth shell 164 may include a light transmission hole 160 positioned to correspond with the location of the light source 159. A light transmission plate 122 may be disposed on the exterior of the fourth shell 164 and covers the light transmission hole 160. During use, the light emitted by the light source 159 passes through the light transmission hole 160 and the light transmission plate 122 to reach the scalp.

In some embodiments, the outer surface of the fourth shell 164 may be provided with a mounting groove that may receive the light transmission plate 122. The light transmission hole 160 may extend through the bottom of the mounting groove, and the light transmission plate 122 may be embedded within the mounting groove to ensure proper alignment, secure attachment, and light transparency. In some embodiments, the battery 154 and the lamp board 158 may be disposed in a stacked configuration along the thickness direction of the second rotating arm 104. The thickness direction of both the battery 154 and the lamp board 158 may be aligned with the overall thickness direction of the second rotating arm 104, thereby enabling efficient use of internal space for arrangement.

The inner wall of the second rotating arm 104 may be further provided with a plurality of support columns 162. The plurality of support columns 162 may be arranged to surround the light transmission hole 160 and the lamp board 158 and may be configured to provide structural support to the battery 154. This arrangement prevents the battery 154 and the lamp board 158 from physically interfering with each other during operation or assembly. In some embodiments, the battery 154 may be clamped between the third shell 152 and the plurality of support columns 162 to secure it in place.

Additionally, the outer surface of the second rotating arm 104 may include a groove corresponding to each of the plurality of support columns 162. The end of each second comb tooth 108 may be inserted into the respective groove. This groove arrangement, which follows the internal layout of the plurality of support columns 162, enhances mechanical stability and avoids weakening the fourth shell 164, which could otherwise occur if the grooves were formed independently and without alignment to internal supports.

Optionally, the plurality of second comb teeth 108 and the second shell 138 may be over-moulded, providing additional strength, sealing, or ease of assembly, depending on the manufacturing process selected.

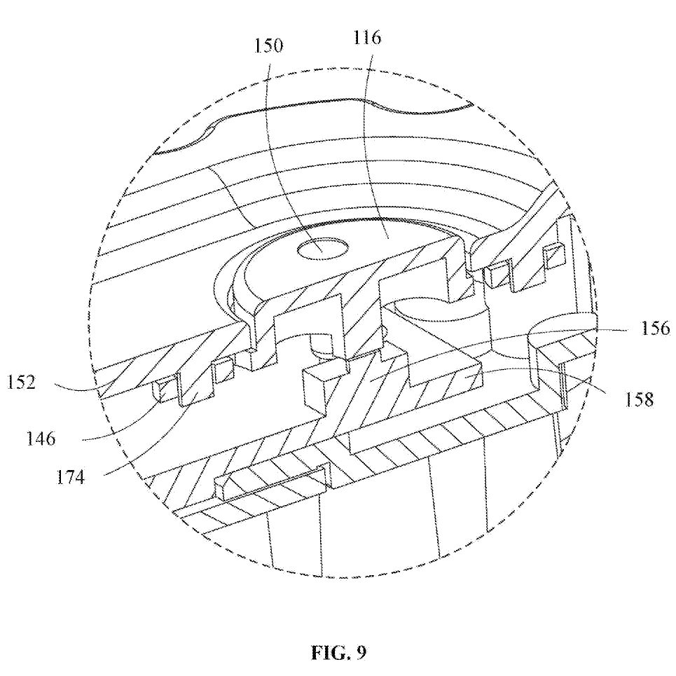

Referring now to FIG. 3 and FIG. 9, in some embodiments, the multifunctional comb 100 may further include a pressing switch 116 and the two charging columns 150. The pressing switch 116 may be provided with two perforations 144, each configured to allow a corresponding charging column 150 to pass through individually. The pressing switch 116 may be operable to trigger a switching device 156 disposed on the lamp board 158.

In another embodiment, the pressing switch 116 is configured to prevent the unintentional activation of the stimulation element. Specifically, the stimulation element is activated only when it receives signals from both the detector unit (such as a sensor or mechanical switch) and the pressing switch 116 simultaneously.

When the pressing switch 116 may be actuated by the user, the pressing switch 116 may activate the switching device 156, thereby controlling the operation of the light source 159, specifically turning the phototherapy light on or off. In addition to this activation function, the pressing switch 116 may also serve as an access point for charging. During charging, the pressing switch 116 may be aligned with an external charging device such that the two charging columns 150 come into electrical contact with corresponding electrodes on the charger. In this position, the battery 154 may receive electrical power through the two charging columns 150, which may be electrically connected to the lamp board 158.

By integrating the two charging columns 150 into the structure of the pressing switch 116, it is only necessary to reserve a single hole or recess in the housing of the multifunctional comb 100 for accommodating the switch assembly. This design significantly reduces the number of external openings in the housing, thereby enhancing the sealing performance and improving the overall aesthetic appearance of the multifunctional comb 100. Additionally, this arrangement minimizes surface area intrusion, leading to a cleaner and more compact product design.

Further, an elastic arm 146 may be arranged on both sides of the pressing switch 116. Each elastic arm 146 may be provided with a positioning hole 148. A corresponding positioning post 174 may be formed on the inner wall of the third shell 152 and may be configured to be inserted into the positioning hole 148 to retain the pressing switch 116 in place and facilitate stable assembly.

The first rotating arm 102 and the second rotating arm 104 may both be elongated in the length direction, and one end of the first rotating arm 102 may be rotatably connected to one end of the second rotating arm 104, thereby enabling folding and unfolding of the multifunctional comb 100 along their longitudinal axes.

In an exemplary implementation, the multifunctional comb 100 may include the first rotating arm 102 and the second rotating arm 104, where the second rotating arm 104 may be rotatably connected to the first rotating arm 102 via the rotating shaft 112. This configuration may enable the multifunctional comb 100 to transition between the folded state, in which the first rotating arm 102 and the second rotating arm 104 may be compactly arranged for portability, and the open state, in which the multifunctional comb 100 may be deployed for grooming and scalp treatment.

The first rotating arm 102 may include a plurality of first comb teeth 110 and the first functional piece 106, which may be implemented, for example, as the fluid conduction module, the massage module, or a microcurrent-based therapy module. In one embodiment, the fluid conduction module may include the reservoir chamber 134 storing a treatment liquid, and the plurality of liquid guide columns 118, each having the liquid conduction channel 170 in communication with the reservoir chamber 134. The occlusion piece 120 may be disposed at the end of each liquid guide column 118 to selectively open or block the liquid flow based on contact with the user’s scalp. The liquid may be dispensed automatically when the occlusion piece 120 is pressed during combing, aided by the internal biasing spring.

The second rotating arm 104 may include a plurality of second comb teeth 108 and the second functional piece 157, such as the phototherapy module. The phototherapy module may include the lamp board 158 and one or more light source 159 arranged to emit therapeutic light through the light transmission hole 160 and the light transmission plate 122. The battery 154 may be housed in the second rotating arm 104 and may be electrically connected to the lamp board 158 to power the light source 159. To enhance the structural integrity, the second rotating arm 104 may include the third shell 152 and the fourth shell 164 that may form the second storage cavity 168. The plurality of support columns 162 may surround the internal components and may align with external grooves to support the plurality of second comb teeth 108. The battery 154 and the lamp board 158 may be arranged in a stacked configuration along the thickness direction of the second rotating arm 104 for efficient use of internal space.

Activation of the stimulation therapy function may be performed by pressing the pressing switch 116 disposed on the second rotating arm 104. In an embodiment, the stimulation therapy may include, but is not limited to, phototherapy, microcurrent stimulation, thermal stimulation, and vibrational massage. The pressing switch 116 may include the two perforations 144 through which the two charging columns 150 extend. When pressed, the pressing switch 116 may engage the switching device 156 on the lamp board 158 to turn the light source 159 on or off. The two charging columns 150 may serve as terminals for external recharging by connecting to the charging electrodes.

In an alternative embodiment, the stimulation therapy function of the multifunctional comb 100 may be automatically performed when the multifunctional comb 100 transitions into the open state. In this configuration, a sensor (not shown) may be disposed within or adjacent to the rotating shaft 112 connecting the first rotating arm 102 and the second rotating arm 104. The sensor may be configured to detect the relative angular displacement or separation between the two rotating arms as the multifunctional comb 100 transitions from the folded state to the open state. When the sensor detects that the first rotating arm 102 and the second rotating arm 104 have moved apart to a predetermined threshold angle or position, the sensor generates a control signal to trigger a mechanical action on the pressing switch 116.

The pressing switch 116 may be disposed on the second rotating arm 104 and may include the two perforations 144 through which the two charging columns 150 extend. Upon receiving the control signal from the sensor, the pressing switch 116 may be engaged, either through direct actuation or solenoid-assisted movement, to depress downward onto the switching device 156 positioned on the lamp board 158. Engagement with the switching device 156 toggles the power state of the light source associated with the stimulation therapy function, thereby automatically initiating the therapy when the multifunctional comb 100 is in the open state.

In an exemplary scenario, the multifunctional comb 100 may be used in a liquid dispensing mode to apply a scalp treatment solution, such as a hair growth serum or a moisturising tonic. The user begins by unfolding the multifunctional comb 100 from its folded state to the open state by rotating the second rotating arm 104 relative to the first rotating arm 102 about the rotating shaft 112.

Before use, the user may fill the reservoir chamber 134 located within the first rotating arm 102 by opening the detachable cover plate 114 that seals the liquid injection port 126. The detachable cover plate 114 may be secured by magnetic attachment or snap-fit engagement and may be removed temporarily to pour in the desired treatment fluid. Upon closing the detachable cover plate 114, the sealing ring 124 ensures leakproof containment of the fluid within the reservoir chamber 134. As the user combs their hair using the plurality of first comb teeth 110, the plurality of liquid guide columns 118, which may also be located on the first rotating arm 102, come into contact with the scalp. Each liquid guide column 118 may include the liquid conduction channel 170 that may be normally sealed by the occlusion piece 120 located at the distal end of the corresponding liquid guide column 118. In this embodiment, the occlusion piece may be configured as a spring-based spherical valve that may protrude slightly beyond the surface of the multifunctional comb 100.

When the occlusion piece 120 may be pressed against the scalp during combing, the pressure overcomes the spring force and causes the occlusion piece 120 to move inward, thereby partially opening the liquid conduction channel 170. This allows the fluid stored in the reservoir chamber 134 to flow through each of the plurality of liquid guide columns 118 and may be dispensed onto the scalp surface. The liquid spreads uniformly due to the combing motion and gravity-assisted flow.

When the occlusion piece 120 may be no longer in contact with the scalp, the internal spring returns it to the sealed position at the narrow end of the conical channel, thereby preventing any further outflow of fluid and eliminating leakage when the multifunctional comb 100 is not in use. In some embodiments, the plurality of liquid guide columns 118 may be arranged to protrude farther than the plurality of first comb teeth 110, thereby ensuring scalp contact and reliable dispensing regardless of hair density.

In another exemplary scenario, the multifunctional comb 100 may be used to perform a stimulation therapy treatment on the user’s scalp. The user first transitions the multifunctional comb 100 from the folded state to the open state by rotating the second rotating arm 104 away from the first rotating arm 102 about the rotating shaft 112, as previously described. The second rotating arm 104 may house the second functional piece 157, implemented in this embodiment as a phototherapy element. The phototherapy module may include the lamp board 158 with at least one light source 159 mounted thereon, powered by the battery 154. At least one light source 159 may be configured to emit therapeutic light, such as red light, infrared light, blue light, or violet light, suitable for rejuvenation, anti-inflammatory treatment, or stimulation of hair follicles.

To initiate the treatment, the user may press the pressing switch 116 disposed on the outer surface of the second rotating arm 104. Upon actuation, the pressing switch 116 may engage the switching device 156 located on the lamp board 158, thereby activating the light source 159. The light emitted from the light source 159 may pass through the light transmission hole 160 formed in the fourth shell 164 of the second rotating arm 104 and may exit through the light transmission plate 122 mounted over the light transmission hole 160. The light transmission plate 122 may be embedded within a mounting groove on the exterior surface of the second rotating arm 104, thereby ensuring proper optical alignment and structural sealing.

The user may gently glide the second rotating arm 104 across the scalp area while the phototherapy light is emitted. The emitted light may interact with the scalp tissue, delivering targeted therapeutic effects such as improving microcirculation, thereby reducing inflammation, and enhancing cellular regeneration. The user may continue the phototherapy treatment for a predefined duration or until the entire scalp area has been covered.

Finally, it should be noted that the above-described embodiments are provided solely for the purpose of illustrating the technical solutions of the present invention and are not intended to limit the scope thereof. Although the present invention has been described in detail with reference to the foregoing embodiments, those skilled in the art will appreciate that various modifications, alterations, and substitutions can be made to the described technical features without departing from the spirit and scope of the present invention. Such variations shall be considered within the scope of protection as defined by the appended claim

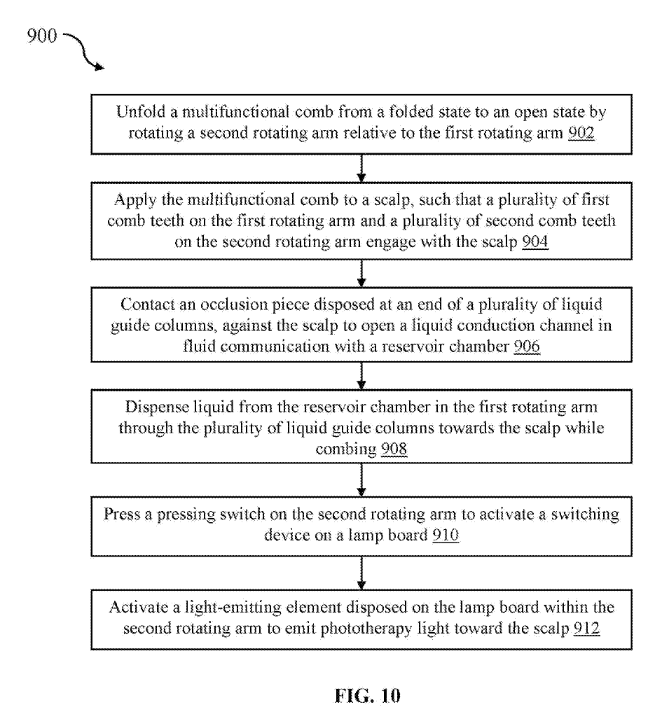

Referring to FIG. 10, a flowchart 900 of a methodology to treat the scalp using the multifunctional comb 100, in accordance with an embodiment of the present disclosure. The flowchart 900 may include a plurality of steps so as to treat the scalp using the multifunctional comb 100.

At step 902, the method begins by unfolding the multifunctional comb 100 from a folded state into an open state. This may be achieved by rotating the second rotating arm 104 relative to the first rotating arm 102 about the rotating shaft 112, thereby allowing the multifunctional comb 100 to be positioned for use. Further, at step 904, the user applies the multifunctional comb 100 to the scalp, such that the plurality of first comb teeth 110 on the first rotating arm 102 and the plurality of second comb teeth 108 on the second rotating arm 104 engage with the scalp surface. This provides simultaneous combing by both the first rotating arm 102 and the second rotating arm 104 during grooming.

Further, at step 906, the method may include contacting the occlusion piece 120 disposed at the end of each liquid guide column 118 against the scalp. This contact may selectively open the liquid conduction channel 170 within each liquid guide column 118, which may be in fluid communication with the reservoir chamber 134 within the first rotating arm 102. Moreover, at step 908, liquid may be dispensed from the reservoir chamber 134 through the plurality of liquid guide columns 118 toward the scalp while combing, enabling delivery of treatment fluids such as hair care or scalp-conditioning solutions directly to the scalp. Further, at step 910, the method proceeds with the user pressing a pressing switch 116 on the second rotating arm 104. The pressing switch 116 may be configured to trigger the switching device 156 on the lamp board 158, which is part of the second functional piece 157.

Further at step 912, the switching device 156 activates the at least one light source 159 disposed on the lamp board 158, causing phototherapy light such as red, infrared, or blue light, to be emitted through the light transmission hole 160 and the light transmission plate 122 located on the second rotating arm 104. This light may be directed toward the scalp to provide therapeutic effects such as anti-inflammation, hair growth stimulation, or scalp rejuvenation.

Thus, the disclosed method and the multifunctional comb 100 try to overcome the technical problem of limited functionality in conventional hair combs, which are typically restricted to mechanical grooming alone. Prior art solutions do not effectively utilize the time during combing to deliver scalp care, nor do they integrate liquid dispensing and stimulation functions in a compact, portable device.

The disclosed method and multifunctional comb 100 address this deficiency by providing a dual-arm, foldable comb structure that supports simultaneous hair grooming and scalp treatment. Through integration of the first functional piece 106 for fluid conduction and the second functional piece 157 for phototherapy, the multifunctional comb 100 enables real-time delivery of care fluids and light-based therapy directly to the scalp during normal combing operations.

As will be appreciated by those skilled in the art, the designs described in the various embodiments discussed above are not routine, or conventional, or well-understood in the art. In light of the above-mentioned advantages and the technical advancements provided by the disclosed method and the multifunctional comb, the claimed steps as discussed above are not routine, conventional, or well understood in the art, as the claimed steps enable the following solutions to the existing problems in conventional technologies. Further, the claimed steps bring an improvement in the functioning of the device itself, as the claimed steps provide a technical solution to a technical problem.

The specification has described a multifunctional comb and a method of treating the scalp using the same. The illustrated steps are set out to explain the exemplary embodiments shown, and it should be anticipated that ongoing technological development will change the manner in which particular functions are performed. These examples are presented herein for the purpose of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

It is intended that the disclosure and examples be considered as exemplary only, with a true scope of disclosed embodiments being indicated by the following claims.

1. A multifunctional comb comprising:
a first rotating arm comprising a plurality of first comb teeth and a first functional piece; and
a second rotating arm comprising a plurality of second comb teeth and a second functional piece,
wherein the second rotating arm is rotatably connected to the first rotating arm to form a folded state and an open state,
wherein in the folded state, the second rotating arm is folded relative to the first rotating arm, and in the open state, the second rotating arm is open relative to the first rotating arm, and
wherein the first functional piece is configured to deliver a liquid to the scalp, and the second functional piece is configured to provide stimulation therapy to the scalp.

2. The multifunctional comb of claim 1, wherein the second functional piece is selected from a group consisting of a microcurrent module, a steamer module, a phototherapy module, an iontophoresis module, a massage module, a thermal heating or cooling element.

3. The multifunctional comb of claim 1, wherein the first rotating arm comprises a reservoir chamber, and the first functional piece comprises a plurality of liquid guide columns, and an occlusion piece,
wherein one end of each of the plurality of liquid guide columns is fixed to the first rotating arm, and the other end protrudes outside the first rotating arm, and
wherein each of the plurality of liquid guide columns has a liquid conduction channel in fluid communication with the reservoir chamber, and the occlusion piece is configured to selectively block and open the liquid conduction channel.

4. The multifunctional comb of claim 3, wherein the first rotating arm comprises a first shell, a second shell, and a comb tooth piece,
wherein the first shell and the second shell define a first storage cavity, and the second shell comprises a plurality of first relinquishing holes, and a plurality of second relinquishing holes,
wherein the comb tooth piece comprises an integrally formed connecting portion and the plurality of first comb teeth, the connecting portion being disposed within the first storage cavity, and the plurality of first comb teeth extending through the plurality of first relinquishing holes, and
wherein the plurality of liquid guide columns extends through the plurality of second relinquishing holes and are provided with limiting flanges that are clamped between the second shell and the connecting portion.

5. The multifunctional comb of claim 4, wherein one side of each of the plurality of second relinquishing holes opposite the connecting portion is provided with a ring of retaining walls, an end of the ring of retaining walls is sealed against an inner wall of the first shell to form the reservoir chamber; and
wherein the first shell further comprises a liquid injection port in communication with the reservoir chamber, the liquid injection port being covered by a detachable cover plate.

6. The multifunctional comb of claim 1, wherein the second functional piece comprises a lamp board and at least one light source disposed on the lamp board.

7. The multifunctional comb of claim 6, wherein the second rotating arm comprises a third shell and a fourth shell forming a second storage cavity that houses a battery and the second functional piece, and
wherein the fourth shell comprises a light transmission hole corresponding to at least one light source, and a light transmission plate is disposed over the light transmission hole on exterior side of the fourth shell.

8. The multifunctional comb of claim 7, wherein the light source is activated based on the folding state of the multifunctional comb, the folded state being determined by a mechanical switch located inside a hinge.

9. The multifunctional comb of claim 7, wherein the at least one light source is a UV light source disposed in the lamp board and is configured to disinfect or sterilize the comb teeth after each use when the multifunction comb is in the folded state.

10. The multifunctional comb of claim 6, further comprising a pressing switch and two charging columns, wherein the pressing switch comprises two perforations through which the two charging columns pass, and wherein the pressing switch being configured to trigger a switching device on the lamp board, and the charging columns being electrically connected to the lamp board.

11. A multifunctional comb, comprising:
a first rotating arm comprising a plurality of first comb teeth; and
a second rotating arm comprising a plurality of second comb teeth;
at least one functional piece in the first rotating arm or the second rotating arm;
wherein the second rotating arm is rotatably connected to the first rotating arm to allow the multifunctional comb to transition between a folded state and an open state;
wherein in the folded state, the second rotating arm is folded relative to the first rotating arm, and in the open state, the second rotating arm is rotated away from the first rotating arm, and
wherein the at least one functional piece is configured to provide stimulation therapy to the scalp and is activated upon detecting that the multifunctional comb is in the open state.

12. A method of treating a scalp using a multifunctional comb, the method comprising:
unfolding the multifunctional comb from a folded state to an open state, wherein the multifunctional comb comprises a first rotating arm and a second rotating arm, wherein the first rotating arm is rotatably connected to the second rotating arm;
applying a plurality of first comb teeth of the first rotating arm and a plurality of second comb teeth of the second rotating arm to the scalp;
dispensing a liquid from a reservoir chamber in the first rotating arm through a plurality of liquid guide columns toward the scalp; and
activating a light source disposed on a lamp board within the second rotating arm, wherein in the open state of the comb activates the light source to provide phototherapy to the scalp, and upon detection of the folded state, a UV light activates to disinfect or sterilize the multifunctional comb.

13. The method of claim 12, wherein dispensing the liquid comprises an occlusion piece disposed at end of each of a plurality of liquid guide columns to selectively open a liquid conduction channel in fluid communication with the reservoir chamber.

14. The method of claim 13, wherein each of the plurality of liquid guide column is supported by a second shell of the first rotating arm and extends through a relinquishing hole, and further comprises a limiting flange clamped between the second shell and a connecting portion of a comb tooth piece.

15. The method of claim 12, wherein the reservoir chamber is formed between a ring of retaining walls and an inner wall of a first shell of the first rotating arm, and the reservoir chamber is refillable via a liquid injection port sealed by a detachable cover plate.

16. The method of claim 12, wherein the light source is disposed on the lamp board and emits light through a light transmission hole in a fourth shell of the second rotating arm.

17. The method of claim 16, wherein the light transmission hole is covered by a light transmission plate disposed on exterior surface of the second rotating arm.

18. The method of claim 17, wherein a battery supplies power to the light source and the battery is supported by a plurality of support columns surrounding the light transmission hole and disposed within a second storage cavity of the second rotating arm.

19. The method of claim 18, wherein ends of the plurality of second comb teeth are inserted into grooves in an outer surface of the second rotating arm, each groove corresponding to one of the support columns.

20. The method of claim 18, further comprising pressing a pressing switch to trigger a switching device on the lamp board and charging the battery via two charging columns aligned with perforations in the pressing switch.
